(19) United States
(12) Patent Application Publication
(10) Pub. No.: US 2023/0310661 A1
(43) Pub. Date: Oct. 5, 2023

(54) OPHTHALMIC DYE

(71) Applicant: Vitreq B.V., Vierpolders (NL)

(72) Inventors: Detlef Gabel, Bremen (DE); Frank Ruseler, Vierpolders (NL)

(73) Assignee: Vitreq B.V., Vierpolders (NL)

(21) Appl. No.: 18/042,779

(22) PCT Filed: Aug. 26, 2021
(86) PCT No.: PCT/EP2021/073564

§ 371 Date: Feb. 24, 2023

(30) Foreign Application Priority Data
Aug. 28, 2020 (EP) 20193304.1

(51) Int. Cl. A61K 49/00; C09B 11/22

(52) U.S. Cl. CPC A61K 49/006; C09B 11/22; A61K 49/0023

(57) ABSTRACT

The present invention relates to the field of medicine, specifically to ophthalmic surgery, more specifically to a novel ophthalmic dye for surgery which does not stain living cells.

### FIELD OF THE INVENTION

The invention relates to the field of medicine, specifically to ophthalmic surgery, more specifically to a novel ophthalmic dye for surgery which does not stain living cells.

### BACKGROUND OF THE INVENTION

Dyes are used frequently in ophthalmological surgery to stain tissues of interest, specifically to enhance the contrast and to make the structures visible which are to be removed during the surgery. In vitreo-retinal surgery, two different structures need to be removed in certain pathological situations in order to restore vision: the epiretinal membrane (ERM) and the internal limiting membrane (ILM). The state of the art is to visualize these structures by staining them with dyes and then to remove them.

Staining of the ERM is often performed with the azo dye Trypan Blue (TB). TB does, however, not stain the ILM strongly [1, 2]. Clinically, only three dyes have been used for ILM staining. The first dye is indocyanine green (ICG). Its spectral properties with an absorption maximum outside the range visible to the eye has limited its usefulness, together with its limited solubility in aqueous solutions such as phosphate-buffered saline (PBS) and balanced salt solutions (BSS) [1]. The second compound, Brilliant Blue G (BBG), used to visualize the ILM [3], does not suffer from this drawback. The third compound, a N-methylated variant of BBG (MBBG), has a lower staining ability compared to BBG. BBG and MBBG are also used in combination with TB.

Several other triphenyl methane dyes have been suggested for ILM staining. Although the ILM is a non-cellular protein layer, the contrast of the staining may be enhanced by the use of dyes which are able to stain dead cells but preferably no living cells [4]. Although BBG has been shown to stain dead cells exclusively [5], most triphenyl methane dyes are (also) able to stain living cells [6].

Especially the staining of living cells is highly problematic, as cells in the retina will be stained, with subsequent possible damage to the cells since the cells will be sensitized by the dye for the intense light used during the surgical procedure. BBG variants such as MBBG display a lower staining ability of the ILM compared to BBG [7]. Hence, there is a continuing need in the art for ILM staining dyes which do not stain living cells and have a high staining ability for the ILM. It is the aim of this invention to provide such dyes.

### SUMMARY OF THE INVENTION

In an aspect, there is provided a compound represented by structure (A), or a pharmaceutically acceptable salt or hydrate thereof,

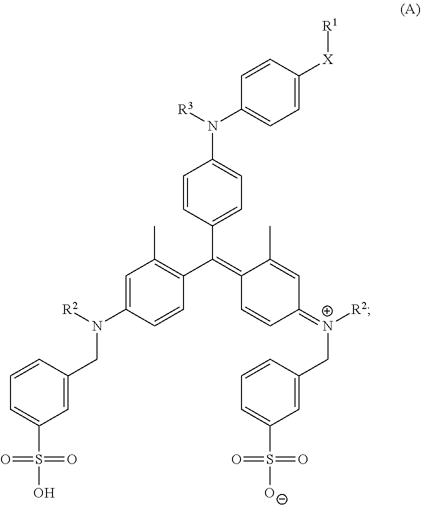

wherein X is O or NH;
wherein R¹ and R² are each independently a C₁₋₈ hydrocarbon or a substituted C₁₋₈ hydrocarbon;
wherein R³ is selected from the group consisting of hydrogen, methyl, a substituted methyl, ethyl and a substituted ethyl; and
wherein at least one of R¹ and R² is a C₃₋₈ hydrocarbon and/or wherein at least one of R¹ and R² is methyl.

Preferably, said compound is for use in a method of ophthalmic surgery, more preferably wherein said ophthalmic surgery comprises staining the internal limiting membrane (ILM) and/or the epiretinal membrane (ERM) and/or within the anterior cavity.

In another aspect, the present invention provides for a composition comprising said compound and a pharmaceutically acceptable excipient.

### DETAILED DESCRIPTION OF THE INVENTION

In a first aspect, there is provided a compound represented by structure (A), or a pharmaceutically acceptable salt or hydrate thereof,

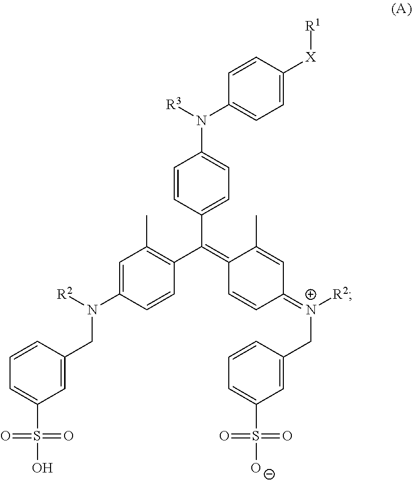

wherein X is O or NH;
wherein R¹ and R² are each independently a C₁₋₈ hydrocarbon or a substituted C₁₋₈ hydrocarbon;
wherein R³ is selected from the group consisting of hydrogen, methyl, a substituted methyl, ethyl and a substituted ethyl; and
wherein at least one of R¹ and R² is a C₃₋₈ hydrocarbon and/or wherein at least one of R¹ and R² is methyl.

Said compound and said pharmaceutically acceptable salt or hydrate thereof are herein referred to as a compound according to the invention. Preferably, a compound according to the invention is for use in a method of ophthalmic surgery, more preferably wherein said ophthalmic surgery comprises staining of an ophthalmic structure, most preferably wherein said ophthalmic structure is the internal limiting membrane (ILM) and/or the epiretinal membrane (ERM) and/or within the anterior cavity. According to this preference, said staining is obtained by contacting said compound according to the invention with a vitreoretinal interface of a subject.

Herein, a hydrocarbon is a chemical moiety consisting of carbon atoms and hydrogen atoms. A Cₓ hydrocarbon is a hydrocarbon, wherein the number of carbon atoms comprised in said hydrocarbon is x. A C_{x-y} hydrocarbon is a hydrocarbon, wherein the number of carbon atoms comprised in said hydrocarbon is from x up to and including y. It is clear to the skilled person that a Cₓ hydrocarbon ora C_{x-y} hydrocarbon may exist as several structural and stereoisomers, all of which are envisioned by the use of “Cₓ hydrocarbon” or “C_{x-y} hydrocarbon”. Hence, without being limiting, a Cₓ hydrocarbon or a C_{x-y} hydrocarbon may be saturated, unsaturated, linear, branched, cyclic, polycyclic or aromatic.

It is further clear to the skilled person that the Cₓ nomenclature may be applied mutatis mutandis to known subsets of Cₓ hydrocarbons. For example, a C₁₋₂ alkyl may be methyl or ethyl. In other words, a C_{x-y} hydrocarbon may be a C_{x-y} alkyl, C_{x-y} cycloalkyl, C_{x-y} alkenyl, C_{x-y} alkynyl, without being limiting. Herein, an alkyl, a cycloalkyl, an alkenyl or an alkynyl may be branched or linear.

A substituted hydrocarbon is a hydrocarbon wherein one or more hydrogen atoms are substituted by a chemical moiety comprising an atom which is not a hydrogen atom and not a carbon atom. Said chemical moieties substituting hydrogen atoms are called substituents herein. The definitions above may be applied mutatis mutandis to substituted hydrocarbons. For example, a substituted C₃ hydrocarbon comprising two chlorine substituents may be 2,3-dichloropropyl, 1,2-dichloropropan-2-yl or 2,2-dichlorocyclopropyl, without being limiting.

In Examples 1 and 2, the synthesis of a compound according to the invention is exemplified. In Example 1, the preparation of compounds (1) and (2) is described. In Example 2, the preparation of compounds (3) to (8) is described. In Table 1 below, these specific compounds according to the invention are defined in terms of their R¹, R² and R³.

TABLE 1
Exemplified compounds according to the invention

| Compound | X | R¹ | R² | R³ |
|---|---|---|---|---|
| (1) | O | iPr | Me | H |
| (2) | O | Bn | Me | H |
| (3) | O | iPr | Et | H |
| (4) | O | nPr | Et | H |
| (5) | O | Bn | Et | H |
| (6) | O | Me | Et | H |
| (7) | O | Ph | Et | H |
| (8) | NH | Ph | Et | H |

In an embodiment, there is provided a compound according to the invention, wherein R¹ is a substituted C₁₋₈ hydrocarbon, and/or R² is a substituted C₁₋₈ hydrocarbon, and/or R³ is selected from the group consisting of a substituted methyl and a substituted ethyl, wherein said substituted C₁₋₈ hydrocarbon, said substituted methyl and/or said substituted ethyl comprise a substituent selected from the group consisting of CF₃, F, CI, Br, I, OH, NH₂, CN, NO and NO₂, more preferably wherein said substituent is CF₃.

In an embodiment, there is provided a compound according to the invention, wherein R¹ and R² are each independently a C₁₋₈ hydrocarbon, and wherein R³ is selected from the group consisting of hydrogen, methyl and ethyl.

In an embodiment, there is provided a compound according to the invention, wherein C₁₋₈ hydrocarbon is selected from the group consisting of C₁₋₈ alkyl, C₃₋₈ cycloalkyl, C₂₋₈ alkenyl, C₂₋₈ alkynyl, phenyl, and benzyl; and/or wherein C₃₋₈ hydrocarbon is selected from the group consisting of C₃₋₈ alkyl, C₃₋₈ cycloalkyl, C₃₋₈ alkenyl, C₃₋₈ alkynyl, phenyl, and benzyl.

In an embodiment, there is provided a compound according to the invention, wherein C₁₋₈ hydrocarbon is selected from the group consisting of methyl, ethyl, n-propyl, i-propyl, n-butyl, phenyl, and benzyl, preferably from the group consisting of methyl, ethyl, n-propyl, i-propyl, phenyl, and benzyl; wherein C₃₋₈ hydrocarbon is selected from the group consisting of n-propyl, i-propyl, n-butyl, phenyl, and benzyl, preferably from the group consisting of n-propyl, i-propyl, phenyl, and benzyl.

30

In an embodiment, there is provided a compound according to the invention, wherein R³ is H.

In an embodiment, there is provided a compound according to the invention, wherein X is O.

In an embodiment, there is provided a compound according to the invention, wherein R² is identical for each instance.

In an embodiment, there is provided a compound according to the invention, wherein said compound is selected from the group consisting of (1), (2), (3), (4), (5), (6), (7) and (8), preferably from the group consisting of (3), (4), (5), (6), (7) and (8), more preferably from the group consisting of (3), (4), (5), (6) and (8), even more preferably from the group consisting of (3), (4), (6) and (8), most preferably wherein said compound is (6) as defined in Table 1 above.

Herein, it is clear that a compounds(1), (2), (3), (4), (5), (6), (7) or (8), or compounds defined by one or more of R¹, R² and R³ in (A), refer to all molecules represented by a structure defined as such and to all pharmaceutically acceptable salts and hydrates thereof. For example, if it is stated that “a compound according to the invention is compound (6)”, said compound may be a compound represented by compound (6) as understood by the skilled person, or any pharmaceutically acceptable salt or hydrate thereof.

In an embodiment, there is provided a compound according to the invention, wherein X is O, R¹ is i-propyl, each R² is ethyl, and R³ is H; or wherein X is NH, R¹ is phenyl, each R² is ethyl, and R³ is H; or wherein X is O, R¹ is n-propyl, each R² is ethyl, and R³ is H; or wherein X is O, R¹ is Me, R² is Et and R³ is H ; or wherein X is O, R¹ is benzyl, each R² is ethyl, and R³ is H. This embodiment corresponds with compounds (3), (4), (5), (6) and (8). In Example 5, it can be seen that such compounds are characterized by a high staining ability of the ILM.

In an embodiment, there is provided a compound according to the invention, wherein X is NH, R¹ is phenyl, each R² is ethyl, and R³ is H; or wherein X is O, R¹ is n-propyl, each R² is ethyl, and R³ is H; or wherein X is O, R¹ is Me, R² is Et and R³ is H; or wherein X is O, R¹ is benzyl, each R² is ethyl, and R³ is H. This embodiment corresponds with compounds (4), (5), (6) and (8). In Example 5, it can be seen that such compounds are characterized by a high staining of the ILM. In an embodiment, there is provided a compound according to the invention, wherein said compound is compound (6) or a pharmaceutically acceptable salt or hydrate thereof.

In a second aspect, there is provided, a composition comprising a compound according to the invention, preferably wherein said compound is compound (6) of a pharmaceutically acceptable salt or hydrate thereof, and a pharmaceutically acceptable excipient. A composition according to this second aspect is herein referred to as a composition according to the invention. Preferably, a composition according to the invention is for use in a method of ophthalmic surgery, more preferably wherein said ophthalmic surgery comprises staining of an ophthalmic structure, most preferably wherein said ophthalmic structure is the internal limiting membrane (ILM) and/or the epiretinal membrane (ERM) and/or within the anterior cavity. According to this preference, said staining is obtained by contacting said compound according to the invention comprised in said composition according to the invention with a vitreoretinal interface of a subject.

In a third aspect, there is provided a kit of parts comprising a composition according to the invention, wherein the kit comprises the compounds comprised in said composition according to the invention within a single composition, or wherein the kit comprises the compounds comprised in said composition according to the invention in multiple compositions. Preferably, the compound according to the invention comprised in said composition according to the invention is compound (6) or a pharmaceutically acceptable salt or hydrate thereof. The kit may further comprise containers, instructions for use and the like. A kit of parts according to this third aspect is herein referred to as a kit of parts according to the invention. Preferably, a kit of parts according to the invention is for use in a method of ophthalmic surgery, more preferably wherein said ophthalmic surgery comprises staining of an ophthalmic structure, most preferably wherein said ophthalmic structure is the internal limiting membrane (ILM) and/or the epiretinal membrane (ERM) and/or within the anterior cavity. According to this preference, said staining is obtained by contacting said compound according to the invention comprised in said composition according to the invention comprised in said kit of parts according to the invention with a vitreoretinal interface of a subject.

As mentioned here above, a compound according to the invention, or a composition according to the invention, or a kit of parts according to the invention is preferably for use as a medicament, more preferably for use in a method of ophthalmic surgery. In a related fourth aspect of the invention is provided a method of treatment, preferably a method of ophthalmic surgery, wherein said method comprised the use of a compound according to the invention, preferably wherein said compound is compound (6) or a pharmaceutically acceptable salt or hydrate thereof, or the use of a composition according to the invention, preferably wherein the compound according to the invention comprised in said composition is compound (6) or a pharmaceutically acceptable salt or hydrate thereof, or the use of a kit of parts according to the invention, preferably wherein the compound according to the invention comprised in said kit of parts is compound (6) or a pharmaceutically acceptable salt or hydrate thereof. In a related fifth aspect of the invention is provided the use of a compound according to the invention, preferably wherein said compound is compound (6) or a pharmaceutically acceptable salt or hydrate thereof, or the use of a composition according to the invention, preferably wherein the compound according to the invention comprised in said composition is compound (6) or a pharmaceutically acceptable salt or hydrate thereof, or the use of a kit of parts according to the invention, preferably wherein the compound according to the invention comprised in said kit of parts is compound (6) or a pharmaceutically acceptable salt or hydrate thereof, for the manufacture of a medicament, preferably a medicament used in ophthalmic surgery.

In an embodiment, there is provided a compound according to the invention, preferably wherein said compound is compound (6) or a pharmaceutically acceptable salt or hydrate thereof, or a composition according to the invention, preferably wherein the compound according to the invention comprised in said composition is compound (6) or a pharmaceutically acceptable salt or hydrate thereof, or a kit of parts according to the invention, preferably wherein the compound according to the invention comprised in said kit of parts is compound (6) or a pharmaceutically acceptable salt or hydrate thereof, for use in a method of ophthalmic surgery, or a method according to the fourth aspect of the invention, or a use according to the fifth aspect of the invention, wherein said ophthalmic surgery comprises staining of an ophthalmic structure, preferably wherein said staining is obtained by contacting the compound according to the invention with a vitreoretinal interface of a subject. Preferably, said ophthalmic structure is the internal limiting membrane (ILM) and/or the epiretinal membrane (ERM) and/or within the anterior cavity. Other preferred ophthalmic structures are selected from the group consisting of the anterior lens capsule, the conjunctiva, the anterior segment, Descemet's membrane and endothelium, and the anterior vitreous. A preferred ophthalmological surgery is selected from the group consisting of cataract surgery, refractive surgery, corneal surgery, vitreo-retinal surgery, and conjunctival surgery, deep lamellar keratoplasty and keratoplasty.

In an embodiment, there is provided a compound according to the invention, wherein said compound is compound (6) or a pharmaceutically acceptable salt or hydrate thereof, or a composition according to the invention comprising a compound according to the invention, wherein the compound according to the invention comprised in said composition is compound (6) or a pharmaceutically acceptable salt or hydrate thereof, or a kit of parts according to the invention comprising such a compound according to the invention, wherein the compound according to the invention comprised in said kit of parts is compound (6) or a pharmaceutically acceptable salt or hydrate thereof, wherein said compound is for use in a method of ophthalmic surgery, wherein said ophthalmic surgery comprises staining of an ophthalmic structure, preferably wherein said staining is obtained by contacting the compound according to the invention with a vitreoretinal interface of a subject, preferably wherein said ophthalmic structure is the internal limiting membrane (ILM) and/or the epiretinal membrane (ERM) and/or within the anterior cavity, wherein said compound does not stain living cells. In the embodiments of the invention, living cells are mammalian cells and are preferably cells comprised in the retina of a subject, more preferably said living cells are Muller glia cells, most preferably said living cells contact said ophthalmic structure in said subject.

Preferably, in the embodiments of the invention, the ability of a compound to stain living or dead cells is determined via the methodology outlined in Example 4. In Example 4, it is confirmed that compound (6) does not stain living cells.

Moreover, the methodology in Example 3, using the dye content in the ARPE19 cells as measured by spectrophotometry as a measure for (visual) staining, provides another test for ability of staining of living cells and dead cells by a dye. Using the methodology of Example 3, a dye with A_{living}<0.07 is considered to be a dye not staining living cells; likewise a dye with A_{dead}<0.07 is considered to be a dye not staining dead cells. More generally, the A_{living} and A_{dead} values obtained via this test may be interpreted as quantitative measures for staining living and dead cells, respectively. In case the methodologies of Examples 3 and 4 present different results for a tested dye, the methodology of Example 4 could be considered as a method for assessing the staining ability of a dye.

From Example 3, it is clear that all exemplified compounds (1) to (8) do not stain living cells (A_{living}≤0.0629). Moreover, it follows from the examples that compounds (1) to (8) show an unexpectedly high ability to stain the ILM relative to the BBG derivatives EBBG, PBBG and BBBG disclosed in the art. Hence, the invention provides a compound for staining an ophthalmic structure, wherein said compound does not stain living cells. As explained above, it is confirmed in Example 4 that compound (6) does not stain living cells.

In an embodiment, there is provided a compound according to the invention, or a composition according to the invention, or a kit of parts according to the invention comprising such a compound according to the invention, wherein said compound, composition or kit is for use in a method of ophthalmic surgery, wherein said ophthalmic surgery comprises staining of an ophthalmic structure, preferably wherein said staining is obtained by contacting the compound or composition according to the invention with a vitreoretinal interface of a subject, preferably wherein said ophthalmic structure is the internal limiting membrane (ILM) and/or the epiretinal membrane (ERM) and/or within the anterior cavity, wherein said compound does not stain living cells, wherein not staining living cells is preferably determined via the methodology provided in Example 4.

In an embodiment, there is provided a compound according to the invention, or a composition according to the invention comprising such a compound according to the invention, or a kit of parts according to the invention comprising such a compound according to the invention, wherein said compound is for use in a method of ophthalmic surgery, wherein said ophthalmic surgery comprises staining of an ophthalmic structure, preferably wherein said staining is obtained by contacting the compound according to the invention with a vitreoretinal interface of a subject, preferably wherein said ophthalmic structure is the internal limiting membrane (ILM) and/or the epiretinal membrane (ERM) and/or within the anterior cavity, wherein said compound has an Ahving lower than 0.1, 0.095, 0.09, 0.085, 0.08, 0.075, 0.07, 0.065, 0.06, 0.055, 0.05, 0.045, 0.04, 0.035, 0.03, 0.025, 0.02, 0.015, or 0.01, wherein said Ahving is determined via the methodology presented in Example 3.

The ability of a compound according to the invention to stain the ILM is an important parameter in the context of the current invention. In an embodiment, there is provided a compound according to the invention, or a composition according to the invention comprising such a compound according to the invention, or a kit of parts according to the invention comprising such a compound according to the invention, wherein said compound is for use in a method of ophthalmic surgery, wherein said ophthalmic surgery comprises staining of an ophthalmic structure, preferably wherein said staining is obtained by contacting the compound according to the invention with a vitreoretinal interface of a subject, preferably wherein said ophthalmic structure is the internal limiting membrane (ILM) and/or the epiretinal membrane (ERM) and/or within the anterior cavity, wherein said compound has a higher ability to stain the ILM comprised in said subject than the corresponding ability of BBG. Herein, (ILM) staining ability and the ability to stain the ILM are used interchangeably.

Preferably, the ability of a compound to stain the ILM is determined via the methodology outlined in Example 5. Herein, a PVDF filter is used as a model for the ILM in a subject. Example 5 demonstrates that compound (6) has an increased ability to stain the ILM compared to BBG, whereas compound (8) demonstrated an increased ability to stain the ILM compared to compound (6).

A compound used in or suitable for the staining of an ophthalmic structure may berein be referred to as an ophthalmic dye or a dye. As explained above, dyes are used frequently in ophthalmological procedures to stain tissues of interest. In ocular surgery of the anterior chamber and the posterior chamber, dyes are being used for enhancing the contrast and to make the structures visible which are to be removed during the surgery. The conditions to be treated in the anterior chamber are, without being exhaustive: deep lamellar keratoplasty dissection, keratoplasty, Descemet's membranes, cataract surgery, conjunctival surgery, and anterior vitreous surgery. In the posterior chamber, conditions are, again without being exhaustive: macular hole, epiretinal membrane, retinal detachment, vitreomacular traction, macular hole development, unroofed cysts, residual membranes, pit-associated maculopathy, retinopathy of prematurity.

A compound according to the invention or a composition according to the invention can conveniently be used in ophthalmic manipulations on a subject such as ophthalmic surgery, preferably as an ophthalmic adjuvant. Examples of diseases and conditions of the eye that require ophthalmic manipulations such as ophthalmic surgery are, but are not limited to, vitreo-retinal disease such as macular hole, retinal detachment due to hymoyopathic macular hole, epiretinal membrane, proliferative diabetic retinopathy, diabetic macular edema, proliferative vitreoretinopathy, specific cataracts such as hypermature cataract, congenital cataract, and split thickness corneal transplantation. The subject is preferably a mammal, more preferably a human.

In vitreo-retinal surgery, two different structures need to be removed in certain pathological situations in order to restore vision: the epiretinal membrane (ERM) (an abnormal structure generated e.g. when the patient suffers from chronic diabetes) and the internal limiting membrane (ILM), which separates the vitreous from the retina, and is firmly attached to the latter.

For surgery, the vitreous is removed, and the eye is filled either with gas or with a salt solution in preparation for removal of the ERM and the ILM from the retina. After staining the ERM and the ILM with one or more dyes, or a composition thereof, these structures are removed. Therefore, a composition according to the invention preferably comprises an additional dye able to stain the ERM. Concentrations of the dyes in a composition according to the invention, i.e. both of the compound according to the invention and of the (one or more) additional dye(s), must be high enough to allow appropriate staining; a reduction in the concentration will usually lead to staining reduced to such low levels that they are of little value for the surgeon.

Besides the concentration of the compound according to the invention and the one or more additional dyes, other preferred parameters of and components comprised in a composition according to the invention may be interesting with regard to the ophthalmological use of said compositions. In this light, several preferred compositions according to the invention will be listed below.

In an embodiment, there is provided a composition according to the invention, wherein the concentration of the compound according to the invention is within the range of about 0.005% (w/v) to about 1% (w/v), more preferably within the range of 0.005% (w/v) to 1% (w/v), more preferably within the range of about 0.01% (w/v) to about 1% (w/v), more preferably within the range of 0.01% to 1%. More preferably, the concentration of the compound according to the invention, is within the range of about 0.005% (w/v) to about 0.05% (w/v), even more preferably within the range of 0.005% (w/v) to 0.05% (w/v). Even more preferably, the concentration of the compound according to the invention, is within the range of about 0.01% (w/v) to about 0.05% (w/v), even more preferably within the range of 0.01% (w/v) to 0.05% (w/v). A preferred concentration is about 0.025%, preferably 0.025%. Preferably, the compound according to the invention comprised in a composition according to this embodiment is compound (6) or a pharmaceutically acceptable salt or hydrate thereof.

In an embodiment, there is provided a composition according to the invention, wherein said composition further comprises an additional dye, preferably wherein said dye is able to stain an ophthalmic structure as defined herein, even more preferably wherein said dye is able to stain the ERM and/or ILM. “Additional” in this context should be interpreted as present besides a compound according to the invention, which may also be called a dye. Hence, the person skilled in the art will comprehend that the composition according to the invention is not limited to a composition comprising a compound according to the invention and a single additional dye; the composition may comprise another additional dye or additional dyes either selected from the dyes listed herein or other dyes. Preferably, the compound according to the invention comprised in a composition according to this embodiment is compound (6) or a pharmaceutically acceptable salt or hydrate thereof.

A preferred dye, being either a compound according to the invention or an additional dye as defined above, has a good staining ability for dead cells, a minimal cellular toxicity in the dark, and a minimal cellular toxicity in the strong illumination necessary for surgery. As set forward here above, Example 5 shows that compounds according to the invention are characterized by a high ability to stain the ILM. In addition, Example 6 shows that compounds (5), (6), (7) and (8) according to the invention have a comparable toxicity relative to BBG, as expressed by the survival of ARPE-19 cells after exposed to the dyes at a concentration of 0.1% in PBS for 15 minutes at 37° C. Specifically, compounds (5), (6), (7) and (8) correspond to a survival of 81%, 103%, 81% and 79% respectively, where BBG corresponds to a survival of 99%.

Preferably, a composition according to the invention comprises an additional dye selected from the group consisting of Trypan Blue, Janus Green, Methyl Green, Methylene Blue, Crystal Violet, Methyl Violet, Ethyl Violet, Evans Blue, Methyl Blue and their respective pharmaceutically acceptable salts and hydrates thereof. Preferably, a composition according to the invention comprises Trypan Blue or a pharmaceutically acceptable salt or hydrate thereof. Preferably, the compound according to the invention comprised in a composition according to this embodiment is compound (6) or a pharmaceutically acceptable salt or hydrate thereof.

Preferably, the concentration of such additional dye selected from the group consisting of Trypan Blue, Janus Green, Methyl Green, Methylene Blue, Crystal Violet, Methyl Violet, Ethyl Violet, Evans Blue, Methyl Blue or a respective pharmaceutically acceptable salt or hydrate thereof, is within the range of about 0.001% to about 1% (w/v), more preferably within the range of 0.001% (w/v) to 1% (w/v), more preferably within the range of about 0.005% (w/v) to about 0.5% (w/v), more preferably within the range of 0.005% (w/v) to 0.5% (w/v), more preferably within the range of about 0.01% (w/v) to about 0.5% (w/v), more preferably within the range of 0.01% (w/v) to 0.5% (w/v), even more preferably within the range of about 0.05% (w/v) to about 0.15% (w/v), even more preferably within the range of 0.05% (w/v) to 0.15% (w/v).

Preferably, in a composition according to the invention:
Janus Green, if present, has a concentration within the range of about 0.001% (w/v) to about 0.1% (w/v), preferably within the range of about 0.005% (w/v) to about 0.01% (w/v),
Methyl Green, if present, has a concentration within the range of about 0.001% (w/v) to about 0.1% (w/v), preferably within the range of about 0.01% (w/v) to about 0.05% (w/v),
Methylene Blue, if present, has a concentration within the range of about 0.001% (w/v) to about 0.1% (w/v), preferably within the range of about 0.01% (w/v) to about 0.1% (w/v),
Crystal Violet, if present, has a concentration within the range of about 0.001% (w/v) to about 0.1% (w/v), preferably within the range of about 0.01% (w/v) to about 0.05% (w/v),
Methyl Violet, if present, has a concentration within the range of about 0.001% (w/v) to about 0.1% (w/v), preferably within the range of about 0.01% (w/v) to about 0.05% (w/v),
Ethyl Violet, if present, has a concentration within the range of about 0.001% (w/v) to about 0.1% (w/v), preferably within the range of about 0.01% (w/v) to about 0.05% (w/v),
Evans Blue, if present, has a concentration within the range of about 0.001% (w/v) to about 0.4% (w/v), preferably within the range of about 0.0025% (w/v) to about 0.1% (w/v),
Indocyanine Green, if present, has a concentration within the range of about 0.001% (w/v) to about 0.4% (w/v), preferably within the range of about 0.0025% (w/v) to about 0.1% (w/v),
Methyl Blue, if present, has a concentration within the range of about 0.001% (w/v) to about 0.4% (w/v), preferably within the range of about 0.0025% (w/v) to about 0.1% (w/v), and,
Trypan Blue, if present, has a concentration within the range of about 0.001% (w/v) to about 0.25% (w/v), preferably within the range of about 0.01% (w/v) to about 0.15% (w/v).

More preferably, in a composition according to the invention:
Janus Green, if present, has a concentration within the range of 0.001% (w/v) to 0.1% (w/v), preferably within the range of 0.005% (w/v) to 0.01% (w/v),
Methyl Green, if present, has a concentration within the range of 0.001% (w/v) to 0.1% (w/v), preferably within the range of 0.01% (w/v) to 0.05% (w/v),
Methylene Blue, if present, has a concentration within the range of 0.001% (w/v) to 0.1% (w/v), preferably within the range of 0.01% (w/v) to 0.1% (w/v),
Crystal Violet, if present, has a concentration within the range of 0.001% (w/v) to 0.1% (w/v), preferably within the range of 0.01% (w/v) to 0.05% (w/v),
Methyl Violet, if present, has a concentration within the range of 0.001% (w/v) to 0.1% (w/v), preferably within the range of 0.01% (w/v) to 0.05% (w/v),
Ethyl Violet, if present, has a concentration within the range of 0.001% (w/v) to 0.1% (w/v), preferably within the range of 0.01% (w/v) to 0.05% (w/v),
Evans Blue, if present, has a concentration within the range of 0.001% (w/v) to 0.4% (w/v), preferably within the range of 0.0025% (w/v) to 0.1% (w/v),
Indocyanine Green, if present, has a concentration within the range of 0.001% (w/v) to 0.4% (w/v), preferably within the range of 0.0025% (w/v) to 0.1% (w/v),
Methyl Blue, if present, has a concentration within the range of 0.001% (w/v) to 0.4% (w/v), preferably within the range of 0.0025% (w/v) to 0.1% (w/v), and,
Trypan Blue, if present, has a concentration within the range of 0.001% (w/v) to 0.25% (w/v), preferably within the range of 0.01% (w/v) to 0.15% (w/v).

Even more preferably, in a composition according to the invention:
Janus Green, if present, has a concentration within the range of about 0.005% (w/v) to about 0.01% (w/v),
Methyl Green, if present, has a concentration within the range of about 0.01% (w/v) to about 0.05% (w/v),
Methylene Blue, if present, has a concentration within the range of about 0.01% (w/v) to about 0.1% (w/v),
Crystal Violet, if present, has a concentration within the range of about 0.01% (w/v) to about 0.05% 40 (w/v),
Methyl Violet, if present, has a concentration within the range of about 0.01% (w/v) to about 0.05% (w/v),
Ethyl Violet, if present, has a concentration within the range of about 0.01% (w/v) to about 0.05% (w/v),
Evans Blue, if present, has a concentration within the range of about 0.025% (w/v) to about 0.1% (w/v),
Indocyanine Green, if present, has a concentration within the range of about 0.025% (w/v) to about 0.1% (w/v),
Methyl Blue, if present, has a concentration within the range of about 0.025% (w/v) to about 0.1% (w/v), and,
Trypan Blue, if present, has a concentration within the range of about 0.01% (w/v) to about 0.15% (w/v).

Even more preferably, in a composition according to the invention:
Janus Green, if present, has a concentration within the range of 0.005% (w/v) to 0.01% (w/v),
Methyl Green, if present, has a concentration within the range of 0.01% (w/v) to 0.05% (w/v),
Methylene Blue, if present, has a concentration within the range of 0.01% (w/v) to 0.1% (w/v),
Crystal Violet, if present, has a concentration within the range of 0.01% (w/v) to 0.05% (w/v),
Methyl Violet, if present, has a concentration within the range of 0.01% (w/v) to 0.05% (w/v),
Ethyl Violet, if present, has a concentration within the range of 0.01% (w/v) to 0.05% (w/v),
Evans Blue, if present, has a concentration within the range of 0.025% (w/v) to 0.1% (w/v),
Indocyanine Green, if present, has a concentration within the range of 0.025% (w/v) to 0.1% (w/v),
Methyl Blue, if present, has a concentration within the range of 0.025% (w/v) to 0.1% (w/v), and,
Trypan Blue, if present, has a concentration within the range of 0.01% (w/v) to 0.15% (w/v).

A more preferred additional dye is Trypan Blue. When the composition according to the invention comprises Trypan Blue as an additional dye, the concentrations are most preferably the following: the compound according to the invention is within the range of about 0.005% (w/v) to about 0.5% (w/v) and Trypan Blue is within the range of about 0.005% (w/v) to about 0.5% (w/v), more preferably the compound according to the invention is within the range of about 0.01% (w/v) to about 0.5% (w/v) and Trypan Blue is within the range of about 0.01% (w/v) to about 0.5% (w/v), more preferably the compound according to the invention is within the range of about 0.025% (w/v) to about 0.1% (w/v) and Trypan Blue is within the range of about 0.05% (w/v) to about 0.15% (w/v). Even more preferably, the compound according to the invention is within the range of 0.005% (w/v) to 0.5% (w/v) and Trypan Blue is within the range of 0.005% (w/v) to 0.5% (w/v), more preferably the compound according to the invention is within the range of 0.01% (w/v) to 0.5% (w/v) and Trypan Blue is within the range of 0.01% (w/v) to 0.5% (w/v), more preferably the compound according to the invention is within the range of 0.025% (w/v) to 0.1% (w/v) and Trypan Blue is within the range of 0.05% (w/v) to 0.15% (w/v).

In an embodiment, there is provided a composition according to the invention, wherein said compound according to the invention is selected from the group consisting of compounds (1), (2), (3), (4), (5), (6), (7) and (8), preferably from the group consisting of compounds(3), (4), (5), (6), (7) and (8), more preferably from the group consisting of compounds (3), (4), (5), (6) and (8), even more preferably from the group consisting of compounds (3), (4), (6) and (8), most preferably wherein said compound is compound (6), wherein said composition further comprises Trypan Blue, wherein the concentration of the compound according to the invention is within the range of 0.025% (w/v) to 0.1% (w/v) and the concentration of Trypan Blue is within the range of 0.05% (w/v) to 0.15% (w/v).

Besides their concentration, the purity of a compound according to the invention and other dyes comprised in a composition according to the invention is preferably as high as possible, preferably of pharmaceutical grade, although the dyes herein are not necessarily limited thereto. Preferably, the purity of a dye (including the compound according to the invention) herein is at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or most preferably at least 99% pure. Preferably, the compound according to the invention comprised in a composition according to this embodiment is compound (6) or a pharmaceutically acceptable salt or hydrate thereof.

Examples of pharmaceutical acceptable salts include but are not limited to, salts formed with organic bases (such as glucosamine, galactosamine, mannosamine, meglumine, trimethylamine, choline, procaine, triethanolamine, diethanolamine, and ethanolamine), inorganic bases (such as ammonia, alkaline metals, and alkaline earth metals), organic acids (such as para-toluene sulfonic acid, methane sulfonic acid, formic acid, trifluoro acetic acid, and maleic acid), inorganic acids (such as hydrochloric acid, sulfuric acid, phosphoric acid and nitric acid), basis amino acids (such as lysine, arginine, histidine and ornithine), halogen ions (such as F and CI ions) and intramolecular salts.

Ophthalmic dye compositions such as a composition according to the invention are typically prepared in commercially available phosphate-buffered saline solution (PBS). Accordingly, a composition according to the invention preferably comprises PBS or is PBS based. When a composition according to the invention comprises an agent that increases the density and/or viscosity of the composition, such as specified here below, PBS may be replaced (in part) by the stock solution of the agent and/or may be replaced in part by water to adjust its osmolality to the desired physiological range, such as specified here below. Alternatively, the composition according to the invention is a non-aqueous liquid and is prepared on the basis of a perfluorocarbon liquid as defined elsewhere herein. Preferably, the compound according to the invention comprised in a composition according to this embodiment is compound (6) or a pharmaceutically acceptable salt or hydrate thereof.

The composition according to the invention can be in any form, such as a mixture of solids and a liquid, preferably an aqueous liquid or a perfluorocarbon liquid. A compound according to the invention is preferably soluble in water or a in watery composition such as a buffer. Alternatively or in combination, the compound according to the invention is soluble in a perfluorocarbon liquid. Preferably, at least 0.1% (w/v) can be dissolved in water, in a watery composition such as a buffer or in a perfluorocarbon liquid, more preferably at least 0.1% (w/v), even more preferably at least 1% (w/v) and most preferably at least 5% (w/v) in water, in a watery composition such as a buffer, or in a perfluorocarbon liquid. All components may be present in a single composition or may be present in different compositions that are mixed before use; all (dye) components may be present in a solid mixture that is dissolved before use; for preparation it may conveniently be dissolved in intraocular cleaning solution, intraocular rinsing solution, physiological saline or a balanced salt solution. The composition according to the invention may comprise or may be mixed with a pharmaceutically acceptable excipient and/or carrier and/or a drug known to the persons skilled in the art. After preparation, the composition according to the invention may be sterilized, e.g. by filtration or autoclaving.

In an embodiment, there is provided a composition according to the invention, wherein said composition comprises or consists of a solution, wherein the pH of said solution is within the range of pH 7.0 to pH 7.6, more preferably in the range of pH 7.1 to pH 7.5, more preferably in the range of pH 7.2 to pH 7.5, more preferably in the range of pH 7.3 to pH 7.5 even more preferably the pH is physiological, i.e. neutral, i.e. about pH 7.4, most preferably the pH is 7.4. Preferably, the compound according to the invention comprised in a composition according to this embodiment is compound (6) or a pharmaceutically acceptable salt or hydrate thereof.

Perfluorocarbon liquids (PCFLs) can be used in ophthalmic surgery. The physical properties of PFCLs including high specific gravity, moderate surface tension, low viscosity, and optical clarity and transparency make them convenient intraoperative tools for ophthalmic surgery.

Accordingly, preferably, a composition according to the invention further comprises a perfluorocarbon liquid (PCFL), preferably a perfluorocarbon liquid selected from the group consisting of perfluorooctane, perfluoroperhydrophenanthrene, perfluorodecalin, perfluorotributylamide and perfluorooctylbromide, more preferably perfluorooctane or perfluorodecalin. The person skilled in the art will comprehend that a PCFL for use in ophthalmic surgery should be as pure as possibly, preferably at least 95%, 96%, 97%, 98%, 99% or 100% pure. The person skilled in the art will comprehend that a composition according to the invention comprising a PCFL and a dye according to the invention will not be a watery composition since a PCFL and water hardly mix. Accordingly, the concentration of a PCFL or the total concentration of PCFL in a composition according to the invention is preferably at least 95%, 96%, 97%, 98%, 99% or 100%.

During ophthalmic manipulations on a subject such as ophthalmic surgery, when the eye is filled with a salt solution, a dye solution such as a composition according to the invention is injected into the salt solution and should then sink, by gravity, onto the retina. Different additives are used to increase density and viscosity of a composition according to the invention for this purpose. One additive is a polymer, such as polyethylene glycol (PEG). PEG has the disadvantage that it reduces the staining and thereby the visibility of the ILM. Other additives are sugars or sugar alcohols. These have the disadvantage that they contribute to the osmotic pressure and thus to cell death if not balanced by a reduction in other osmotically active substances. Therefore, their maximum concentration, and as a consequence the density of the solution which can be applied safely, is limited. More importantly, the staining effect of the dye can be reduced severely by some of these additives, especially polymeric additives.

Preferably, an additive comprised in a composition according to the invention, added to increase the density and/or viscosity of said composition, should not reduce the staining which is obtained by contacting the dyes comprised in said composition with a vitreoretinal interface of said subject. More preferably, an additive for increasing the density enhances the staining. In this context, the staining may be measured using the methodology of Example 3.

Preferably, a composition according to the invention exhibits low toxicity. According to a preferred definition, a low toxicity means that at least 60%, 65%, 70%, 75%, 80%, 85%, 90% or 95% cell viability is retained after incubation in PBS for 15 minutes at 37° C. and 5% CO₂, as determined using the methodology of Example 6.

In an embodiment, there is provided a composition according to the invention, wherein said composition further comprises an agent that that increases the density and/or viscosity of the composition, such that the density and/or viscosity of the composition is increased compared to the density and/or viscosity of H₂O when measured under identical conditions. Such agent is preferably selected from the group consisting of heavy water, diglycerol, triglycerol, a Gadolinium complex, a sugar, a sugar alcohol, a polymer and a polysaccharide, more preferably such agent is selected from the group consisting of diglycerol, triglycerol and a Gadolinium complex, most preferably such agent is diglycerol. Preferably, the compound according to the invention comprised in a composition according to this embodiment is compound (6) or a pharmaceutically acceptable salt or hydrate thereof.

In an embodiment, there is provided a composition according to the invention, wherein said composition further comprises diglycerol and/or a dye, preferably wherein said dye is trypan blue.

Preferably, in a composition according to the invention the total concentration of the diglycerol and/or triglycerol, if present, is within the range of about 0.1% to about 25% (v/v), preferably within the range of about 2% to about 20% (v/v), more preferably within the range of about 3% to about 6% (v/v). The physiologically normal osmotic pressure is around 300 mosmol/kg. Accordingly, the osmotic pressure of the composition according to the invention is preferably within the range of about 250 mosmol/kg to about 400 mosmol/kg, more preferably from about 275 mosmol/kg to about 350 mosmol/kg, more preferably from about 285 mosmol/kg to about 350 mosmol/kg. Preferably, the compound according to the invention comprised in a composition according to this embodiment is compound (6) or a pharmaceutically acceptable salt or hydrate thereof.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less about 10% of the value.

All patent and literature references cited in the present specification are hereby incorporated by reference in their entirety.

### FIG. LEGENDS

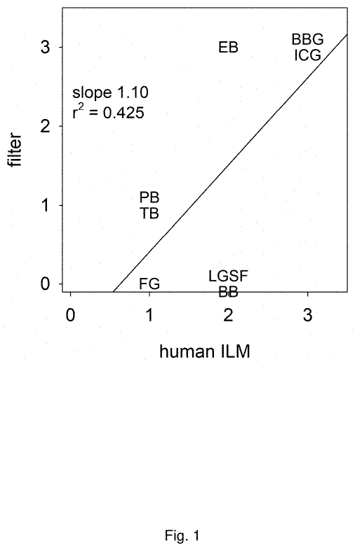

FIG. 1. Correlation of staining of human ILM (data from [8]) and PVDF filter (own data). BB: Bromophenol Blue; BBG: Brilliant Blue G; EB: Evans Blue; FG: Fast Green; ICG: Indocyanine Green; LGSF: Light Green SF; PB: Patent Blue; TB: Trypan Blue

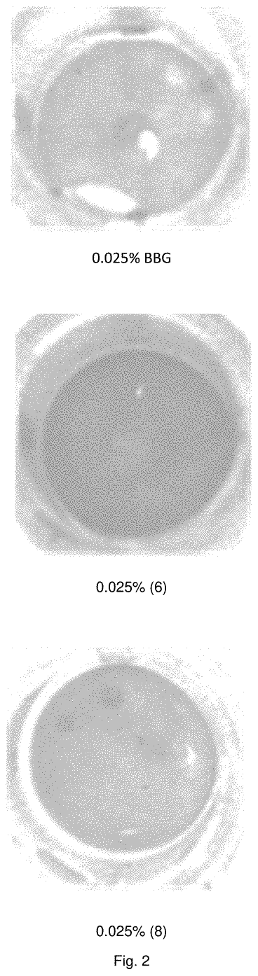

FIG. 2. Staining of PVDF filter by BBG and by three of the dyes according to the invention. The concentrations of the dyes are given in % (weight/volume).

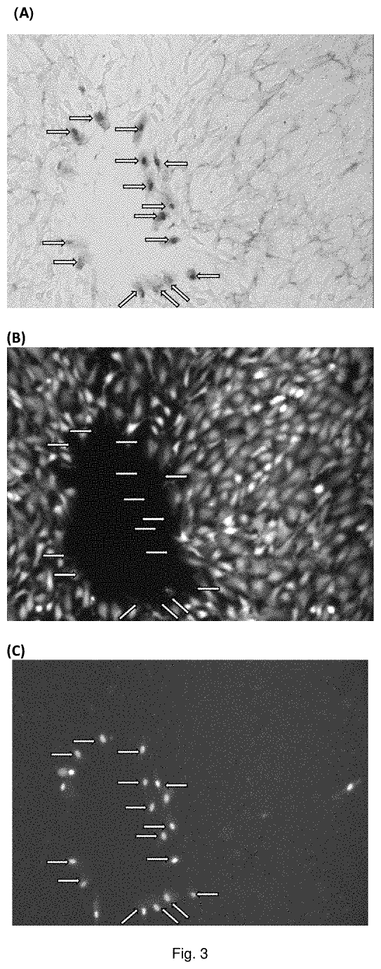

FIG. 3. Photograph with stained cells pointed out by arrows (a), photograph with 485 nm/535 nm for calcein (b), photograph with 530 nm/620 nm for propidium iodide (c).

### EXAMPLES

The present invention is further described by the following examples which should not be construed as limiting the scope of the invention.

### Example 1

### Preparation of Compounds (1) and (2)

N-Methyl-m-toluidine (17.65 g) was reacted with benzyl bromide (18 mL) in 150 mL acetonitrile and 30 g K₂CO₃for 24 h. The reaction solution was freed from the solids by filtration and mixed with 200 mL ethyl acetate. Water (100 mL) was added, and the organic phase was separated and washed with water. The solvent was removed by evaporation, yielding 27.93 g of N-benzyl-N-methyl-m-toluidine.

The product was dissolved in 120 mL concentrated sulfuric acid, and 17 mL fuming sulfuric acid (60% SO₃) was added at 0° C. The mixture was heated to 100° C. overnight, and then poured on ice. After neutralization with NaOH, solids were removed by filtration, and water was removed by lyophilization. The residue was extracted with 200 mL methanol to yield 3-((methyl(m-tolyl)amino)methyl)benzenesulfonic acid.

The sulfonic acid (9.54 g) was condensed with p-chlorobenzaldehyde (2.34 g) in a mixture of water (180 mL) and ethanol (60 mL), and the pH was adjusted to 0.7 with concentrated HCl. The reaction was heated to 120° C. for 3 days.

After cooling, 1.07 g K₂Cr₂O₇ (dissolved in 10 mL of water) was added to the reaction flask, and then 1.79 g oxalic acid (dissolved in 20 mL of water). After 1 h, 25 g of Na₂SO₄ was added. The aqueous layer was mixed with 200 mL butanol, the butanol phase was separated, and the solvent was removed by rotary evaporation.

A part (0.60 g) of the green dye obtained was used without further purification for condensation with 4-iosopropoxyaniline (500 μL) in 20 mL butanol and refluxed for 24 h. After cooling, the solution was poured into 200 mL diethyl ether, the solid was recovered by centrifugation, and subjected to column chromatography on silica gel with a gradient of dichloromethane and methanol. The dye-containing fractions were pooled.

The synthesis described in this example yields compound (1). The compound (2) was obtained in the same way, using p-anisidine in the last step.

### Example 2

### Preparation of Compounds (3) to (8)

In a round-bottom flask with stirrer and reflux condensor, 3.0 g (0.01 mol) 3-((ethyl(m-tolyl)amino)methyl)benzenesulfonic acid and 4-chlorobenzaldehyde (0.005 mol, 0.7 g) were 40 dissolved in a mixture of 50 mL water and 20 mL ethanol. The pH value was adjusted to pH=1.7 with HCl. The reaction mixture was refluxed for 72 hours. The solvent was removed by rotary evaporation, and the residue was dissolved in 30 mL acetonitrile.

Ce(IV)(NH₄)₂(NO₃)₆ (2.3 g, dissolved in 10 mL of a KCl/HCl solution adjusted to pH =1.8) was 5 added under stirring. After 1 hour, the dye was extracted into 100 mL 1-butanol and the solvent was removed by rotary evaporation.

The raw product was dissolved in 50 mL 1-butanol in a round-bottom flask equipped with stirrer and reflux condensor. 4-Isopropoxyaniline (0.01 mol =1.35 mL) was added, and the solution was refluxed for 18 hours. After cooling to room temperature, the mixture was poured into 500 mL diethylether, and the precipitate was collected by centrifugation. It was chromatographed on silica gel with a linear gradient of dichloromethane and methanol. The fractions containing the blue dye were pooled.

15 The synthesis described in this example yields compound (3). The compounds (4), (5), (6), (7), and (8) were obtained in the same way, using p-propyloxyaniline, p-benzyloxyaniline, p-anisidine, p-phenoxyaniline, and 4-aminodiphenylamine, respectively.

### Example 3

### Assessment of Staining of Dead Cells and/or of Living Cells

ARPE19 cells (a human retinal pigment epithelial cell line) were grown in 96-well plates. In half of the wells, cell death was induced by fixation with 70% ethanol for 5 minutes.

Assessing staining of living cells and of dead cells with dyes was carried out with solutions of the dyes (all in a concentration 0.1% weight per volume, except where indicated differently) in PBS (ICG was dissolved in water at 0.5% and diluted to 0.1% with 5% glucose solution). The duration of staining of the cells was 15 minutes. After the incubation, the wells were washed with PBS. A mixture of DMSO (90%) and PBS (10%) was used to lyse the cells and solubilize the dyes.

The dye content in the ARPE19 cells as measured by spectrophotometry was used as a measure for (visual) staining. A complete spectrum from 400 to 1000 nm was recorded. The absorbance from cells only was subtracted, and the minimum absorbance was set to 0. The maximum absorbance for dead cells (A_{dead}) and for living cells (A_{living}) was recorded, and interpreted as measures for the staining of dead and living cells, respectively.

Absorbances below 0.07 were considered too weak to assume that staining can be visualized. Hence, dyes for which A_{living}<0.07 can be considered as dyes which do not stain living cells.

TABLE 2
Staining of living cells and dead cells
by dyes represented by structure (A)

| Compound | X | R¹ | R² | R³ | A_{living} | A_{dead} |
|---|---|---|---|---|---|---|
| (1) | O | iPr | Me | H | 0.0283 | 0.3481 |
| (2) | O | Bn | Me | H | 0.0253 | 0.2454 |
| (3) | O | iPr | Et | H | 0.0440 | 0.6387 |
| (4) | O | nPr | Et | H | 0.0485 | 0.8084 |
| (5) | O | Bn | Et | H | 0.0327 | 0.6734 |
| (6) | O | Me | Et | H | 0.0603 | 0.8755 |
| (6)* | O | Me | Et | H | 0.0635 | 0.3630 |
| (7)* | O | Ph | Et | H | 0.0216 | 0.1976 |
| (8) | NH | Ph | Et | H | 0.0629 | 0.8411 |
| BBG | O | Et | Et | H | 0.0328 | 0.5651 |
| MBBG | O | Et | Et | Me | 0.0355 | 0.6876 |
| EBBG | O | Et | Et | Et | 0.0724 | 0.9066 |
| PBBG | O | Et | Et | Pr | 0.1331 | 0.7369 |
| BBBG | O | Et | Et | Bu | 0.1491 | 0.7941 |

*Concentration of the dye: 0.025%

TABLE 3
Staining of living cells and of dead cells by various dyes

| Compound | A_{living} | A_{dead} |
|---|---|---|
| Acid Violet | 0.0184 | 0.2077 |
| Aniline Blue | 0.0080 | 0.0057 |
| BBR1 | 0.0273 | 0.1467 |
| Brilliant Green | 0.1088 | 0.0568 |
| Bromophenol Blue | 0.0059 | 0.0357 |
| Crystal Violet | 0.5797 | 0.4365 |
| Eosin | 0.0362 | 0.2358 |
| ICG | 0.0703 | 1.1751 |
| Light Green SF | 0.0065 | 0.0080 |
| Malachite Green | 0.0058 | 0.0068 |
| Methyl Green | 0.0489 | 0.0750 |
| Methyl Violet | 1.2503 | 0.8900 |
| Patent Blue 1 | 0.0155 | 0.0113 |
| Trypan Blue | 0.0135 | 0.0847 |

All compounds in Table 2 may be represented by Structure (A), and may be referred to as BBG derivatives herein. Compounds (1)-(8) are compounds according to the invention. BBG, EBBG, PBBG and BBBG are compounds known in the art.

Clinically used BBG does not stain livings cells (A_{living}=0.0328). The staining of dead cells can be increased by substituting R³ by longer alkyl groups in EBBG (R³=Et, A_{dead}=0.9066), PBBG (R³=Pr, A_{dead}=0.7369) and BBBG (R³=0.7941, A_{dead}=0.7941). However, in the search for a BBG derivative which have an increased ability to stain the ILM, this substitution pattern comes at a cost. The staining of living cells increases with increasing size of the R³ group (A_{living}=0.0724 for EBBG; 0.1331 for PBBG; 0.1491 for BBBG). Hence, the art suggests that increasing the number of carbon atoms comprised in R¹, R² or R³ does not yield compounds which do no stain living cells.

The current invention unexpectedly provides a new set of compounds, exemplified but not limited 10 to (1)-(8), wherein the number of which compounds have increased ability for staining the ILM and which do not stain living cells. Indeed, for all (1)-(8) A_{living}<0.07. The structural difference between the BBG derivatives known in the art (BBG, EBBG, PBBG, BBBG) and the compounds according to the inventions is the variation of R¹ and R² relative to BBG in (1)-(8) instead of the variation in R³ in BBG, EBBG, PBBG, BBBG.

As explained, the art seems to suggest that increasing the number of carbon atoms in R¹, R² and R³ would lead to an increased staining of living cells. Interestingly, the compounds according to the invention wherein one of R¹ and R² has more than two carbon atoms and the other one has exactly two carbon atoms ((3), (4), (5), (7), (8)), have a high staining ability. In other words, the compounds wherein R¹ or R² has been elongated with respect to the ethyl group in BBG, show a high staining but do not stain living cells. This set of compounds is a preferred subset of the compounds according to the invention.

All non-BBG dyes tested and listed in Table 3 show either staining of living cells with A_{living}>0.07 (Brilliant Green, Crystal Violet, ICG, Methyl Violet), or are characterized by a relatively low ability of staining ophthalmic structures.

In conclusion, using the dye content in the ARPE19 cells as measured by spectrophotometry as do not stain living cells.

### Example 4

### Staining by Compound (6)

An experiment was carried out with ARPE-19 cells and staining with compound (6). The aim of the experiment was to confirm that compound (6) does not stain living cells.

A three-step staining procedure was used: first the dye was introduced, then calcein-acetoxymethyl ester (calcein-AM), then propidium iodide (PI). Calcein-AM (which is non-fluorescent) is able to penetrate cell membranes and is hydrolyzed by intracellular esterases to calcein, which is fluorescent and which cannot escape through the intact cell membrane. Cells fluorescent for calcein-AM are therefore intact and living cells. Dead cells (cells with a compromised cell membrane) might still hydrolyze calcein-AM, but they are not able to retain the calcein. Cells with compromised cell membranes can take up propidium iodide, a dye which intercalates into DNA and turns fluorescent upon binding.

Cells were grown in 48-well plates. Growth medium was removed and a solution of 20% ethanol was added for 5 minutes in order to induce cell death in some cells. The ethanol solutions were removed. Staining with compound (6) (0.025%) in PBS was carried out at room temperature for 15 minutes. The dye was washed off with PBS. The cells were then exposed to calcein-acetoxymethyl ester (calcein-AM) in PBS for 15 minutes at room temperature. After a wash with PBS, the cells were then exposed to PI for 5 minutes, and microphotographs were taken with a black-and-white camera, using a halogen lamp for illumination for the picture showing the stain (FIG. 3(a)), and with a xenon lamp and appropriate filter combinations for the two fluorescent dyes (FIGS. 3(a) and 3(b)).

The picture with the stain was overlayed with arrows pointing toward the stained cells, and the overlay was then (with maintained x-y coordinates) overlayed on the fluorescent pictures.

From FIG. 3 it clearly follows that compound (6) is does not stain living cells.

### Example 5

### Staining Ability

A PVDF filter has been found to represent the best non-physiological model of the ILM in patients. Correlation between staining of the filter and observed staining of the ILM in patients (data from (Rodrigues, Penha et al. 2010)) show a good correlation (see FIG. 1).

A 96-well filter (MSBVN1210 MultiScreen BV Filter Plate, Merck-Millipore, Darmstadt, Germany) was used as ILM model. The filter material is hydrophilic polyvinylidene difluoride (PVDF). A Collagen Cell Carrier membrane (CCC) (Viscofan, Weinheim, Germany) was used as model for the ERM.

Punches of the membrane (5 mm diameter) were fixed to the filter with a hypodermic needle and soaked in PBS for 30 minutes. The PBS was removed by vacuum filtration, and 50 μL of the dye solution was pipetted into the filter well. After 30 s, the dye was removed by vacuum filtration, and membrane and filter were washed three times with PBS. The membrane was removed and put into a 96-well plate for spectrum recording, using a Tecan Spark 20M (Tecan Group Ltd., Männedorf, Switzerland) plate reader.

The filter staining of the compounds (6) and (8) according to the invention are shown in FIG. 2 in comparison to that of BBG. Much stronger staining is observed for compound (6) than for BBG for equal concentrations of 0.025%, and even stronger staining is observed with compound (8) at 0.005%. The intensity of the grey values was measured. For BBG the mean intensity was 201, for compound (6) it was 175, for compound (8) it was 197. On the scale used, 255 is white and 0 is black.

### Example 6

### Toxicity

The dyes were tested for toxicity as described in the literature [9] at concentrations which stain the filter at an intensity comparable to or surpassing the staining achieved with BBG at 0.025% (the concentration used clinically for this dye). The results are listed in Table 4.

TABLE 4
The survival of the cells after exposure to dyes is given in percent

| Compound | Concentration (%) | Survival (%) |
|---|---|---|
| BBG | 0.025 | 99 |
| (5) | 0.025 | 81 |
| (6) | 0.025 | 103 |
| (7) | 0.025 | 81 |
| (8) | 0.025 | 79 |
| BBG | 0.025 | 99 |
| EBBG | 0.025 | 93 |
| PBBG | 0.005 | 66 |
| BBBG | 0.005 | 67 |

### REFERENCE LIST

1. Semeraro F, Morescalchi F, Duse S, Gambicorti E, Russo A, Costagliola C (2015) Current Trends about Inner Limiting Membrane Peeling in Surgery for Epiretinal Membranes. J Ophthalmol 2015: Article ID 671905
2. Yamamoto N, Ozaki N, Murakami K (2004) Double Visualization Using Triamcinolone Acetonide and Trypan Blue during Stage 3 Macular Hole Surgery. Ophthalmologica 218: 297-305
3. Veckeneer M, Mohr A, Alharthi E, Azad R, Bashshur Z F, Bertelli E, Bejjani R A, Bouassida B, Bourla D, Crespo I C, Fahed C, Fayyad F, Mura M, Nawrocki J, Rivett K, Scharioth G B, Shkvorchenko D O, Szurman P, Van Wijck H, Wong I Y, Wong D S, Frank J, Oellerich S, Bruinsma M, Melles GR (2013) Novel ‘heavy’ dyes for retinal membrane staining during macular surgery: multicenter clinical assessment. Acta Ophthalmol 10.1111/aos.12208
4. logannsen, M. G. (1977). “Some structural features of vital dyes.” Bulletin of Experimental Biology and Medicine 83(4): 591-595.
5. Hisatomi, T., S. Notomi, T. Tachibana, S. Oishi, R. Asato, T. Yamashita, Y. Murakami, Y. Ikeda, H. Enaida, T. Sakamoto and T. Ishibashi (2015). “Brilliant Blue G double staining enhances successful internal limiting membrane peeling with minimal adverse effect by low cellular permeability into live cells.” Retina 35(2): 310-318.
6. EP1553984B2
7. Beccari, M., E. Bettini and P. Signori (2016). “Dye molecule and dye preparations, in particular for use in surgical methods of ophthalmic surgery and for dyeing proteins”. WO 2016/189475 A1
8. Rodrigues, E. B., F. M. Penha, E. de Paula Fiod Costa, M. Maia, E. Dib, M. J. Moraes, C. H. Meyer, O. J. Magalhaes, G. B. Melo, V. Stefano, A. B. Dias and M. E. Farah (2010). “Ability of new vital dyes to stain intraocular membranes and tissues in ocular surgery.” Am J Ophthalmol 149(2): 265-277.
9. Awad, D., J. Wilinska, D. Gousia, X. Shi, J. Eddous, A. Muller, V. Wagner, C. Hillner, W. Brannath, A. Mohr and D. Gabel (2018). “Toxicity and phototoxicity in human ARPE-19 retinal pigment epithelium cells of dyes commonly used in retinal surgery.” European Journal of Ophthalmology 28(4): 433-440.

1. A compound represented by structure (A), or a pharmaceutically acceptable salt or hydrate thereof, 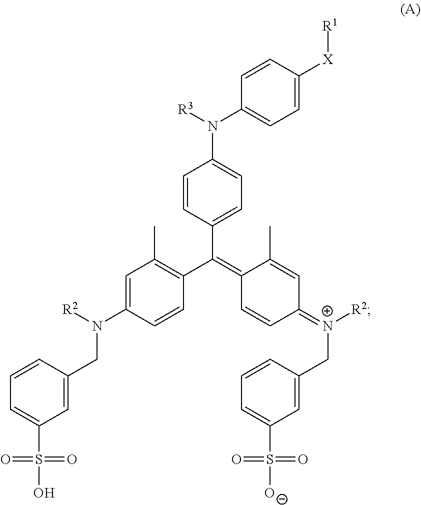
wherein X is O or NH;
wherein R¹ and R² are each independently a C₁₋₈ hydrocarbon or a substituted C₁₋₈ hydrocarbon;
wherein R³ is selected from the group consisting of hydrogen, methyl, a substituted methyl, ethyl and a substituted ethyl; and
wherein at least one of R¹ and R² is a C₃₋₈ hydrocarbon or a substituted C₃₋₈ hydrocarbon and/or wherein at least one of R¹ and R² is methyl or a substituted methyl.

2. The compound according to claim 1, wherein R¹ and R² are each independently a C₁₋₈ hydrocarbon, and wherein R³ is selected from the group consisting of hydrogen, methyl and ethyl.

3. The compound according to claim 1, wherein C₁₋₈ hydrocarbon is selected from the group consisting of C₁₋₈ alkyl, C₃₋₈ cycloalkyl, C₂₋₈ alkenyl, C₂₋₈ alkynyl, phenyl, and benzyl; and/or wherein C₃₋₈ hydrocarbon is selected from the group consisting of C₃₋₈ alkyl, C₃₋₈ cycloalkyl, C₃₋₈ alkenyl, C₃₋₈ alkynyl, phenyl, and benzyl.

4. The compound according to claim 1, wherein C₁₋₈ hydrocarbon is selected from the group consisting of methyl, ethyl, n-propyl, i-propyl, n-butyl, phenyl, and benzyl, preferably from the group consisting of methyl, ethyl, n-propyl, i-propyl, phenyl, and benzyl; wherein C₃₋₈ hydrocarbon is selected from the group consisting of n-propyl, i-propyl, n-butyl, phenyl, and benzyl, preferably from the group consisting of n-propyl, i-propyl, phenyl, and benzyl.

5. The compound according to claim 1, wherein R³ is H.

6. The compound according to claim 1, wherein X is O.

7. The compound according to claim 1, wherein R² is identical for each instance.

8. The compound according to claim 1,
wherein X is NH, R¹ is phenyl, each R² is ethyl, and R³ is H; or
wherein X is O, R¹ is benzyl, each R² is ethyl, and R³ is H; or
wherein X is O, R¹ is methyl, each R² is ethyl, and R³ is H; or
wherein X is O, R¹ is n-propyl, each R² is ethyl, and R³ is H.

9. A composition comprising a compound according to claim 1, and a pharmaceutically acceptable excipient.

10. The composition according to claim 9, wherein said composition further comprises diglycerol and/or a dye, preferably wherein said dye is trypan blue.

11-15. (canceled)

16. A method for ophthalmic surgery comprising staining an ophthalmic structure with the compound of claim 1.

17. The method according to claim 16, wherein said staining is obtained by contacting the compound according to claim 1 with a vitreoretinal interface of a subject.

18. The method according to claim 16, wherein said compound is does not stain living cells.

19. The method according to claim 16, wherein said ophthalmic structure is the internal limiting membrane (ILM) and/or the epiretinal membrane (ERM) and/or within the anterior cavity.
